(19) United States
(12) Patent Application Publication
(10) Pub. No.: US 2005/0268908 A1
(43) Pub. Date: Dec. 8, 2005

(54) Medicament dispenser

(76) Inventors: Stanley George Bonney, Ware (GB); Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/522,325

(22) PCT Filed: Jul. 23, 2003
(86) PCT No.: PCT/EP03/08144

§ 371 Date: Jan. 25, 2005

(30) Foreign Application Priority Data
Jul. 25, 2002 (GB) 0217199.9

(51) Int. Cl. B05D 7/14; A61M 15/00; A61M 16/00; B65D 83/06

(52) U.S. Cl. 128/203.15

(57) ABSTRACT

A medicament dispenser device for use in the delivery of a combination medicament product is disclosed. The device includes a first medicament container for containing a first medicament component; first release means for releasing the contents of the first medicament container; at least one further medicament container for containing at least one further medicament component; at least one further release means for releasing the contents of each the at least one further medicament container; and mixing means for promoting the mixing of the released contents of the first and at least one further medicament container. The first medicament component is kept separate from the at least one further medicament component until the point of release thereof for delivery in combination.

### TECHNICAL FIELD

The present invention relates to a medicament dispenser for dispensing medicament combination products. The invention particularly relates to a device for use in promoting the mixing of previously separately contained multi-active combination medicament products on delivery thereof.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include those in which the medicament carrier is a blister strip containing a number of discrete doses of powdered medicament. Such devices usually contain a mechanism of accessing these doses, usually comprising either piercing means or means to peel a lid sheet away from a base sheet. The powdered medicament can then be accessed and inhaled. Other known devices include those in which the medicament is delivered in aerosol form, including the well-known metered dose inhaler (MDI) delivery devices. Liquid-based inhaler devices are also known.

Therapies involving combinations of different and complementary active medicaments are known. These can be administered either as distinct combination (i.e. multi-active) medicament products, which comprise a defined mixture of each component medicament, or as groups of single active medicament products, which are designed to be taken in combination or sequentially. Whilst combination products offer added convenience for the patient, certain medicament actives are difficult to formulate as distinct combination products. For example, the actives may interact chemically with each other in an undesirable way when formulated together.

It is thus, desirable in certain circumstances, to have a medicament dispenser that separately (i.e. in isolated fashion) contains each active component of a combination product, but which enables the delivery of a combined dose in response to a minimum number of patient actions. In particular, it is desirable that each active component of the combined dose is delivered to the patient in a single, combined dose in response to a single patient dosing action. For example, it is desirable that a combined product for inhalation be delivered in response to a single patient actuation of an inhaler, even where the active components of that combined product are separately stored within the inhaler device.

The Applicants have now appreciated that it can also be desirable to ensure effective mixing of the various active components of the combination product prior to its delivery to the patient. Said mixing advantageously occurs at the point of delivery of the combination product to the patient (i.e. after release from isolated containment but prior to patient delivery) and as an integral part of the medicament release/delivery process rather than by a step-wise process involving a defined ‘pre-mixing’ step followed by a separate ‘delivery’ step. In particular, the mixing is also enabled by the energy inherent in the medicament release/delivery process. For example, such energy can result from an active medicament release (e.g. firing an MDI or aerosolising a liquid spray product) or in the case of an inhaled product, as a result of patient inhalation. Medicament dispenser devices providing such mixing characteristics are thus, herein provided.

The Applicants have also observed that particular medicaments can be more suited to delivery to by particular types of inhaler device. For example, one particular medicament may be more suitable for delivery by an MDI device, whereas another may be more suitable for delivery by a DPI device. That suitability may for example, be driven by ease of formulation of the medicament for that particular inhaler device or by the delivery and pharmaceutical performance characteristics obtainable when the particular inhaler device is employed.

### SUMMARY OF THE INVENTION

According to one aspect of the invention there is provided a medicament dispenser device for use in the delivery of a combination medicament product, the device comprising
a first medicament container for containing a first medicament component;
first release means for releasing the contents of said first medicament container;
at least one further medicament container for containing at least one further medicament component;
at least one further release means for releasing the contents of each said at least one further medicament container; and
mixing means for promoting the mixing of the released contents of the first and at least one further medicament container,
wherein the first medicament component is kept separate from the at least one further medicament component until the point of release thereof for delivery in combination.

The mixing means comprises features and/or components and/or shaping that promote mixing of the released contents of the first and at least one further medicament container, to form a ‘mixed’ multi-active combination product.

Preferably, the mixing means promotes mixing of the released contents immediately prior to their delivery as the combination product, such as at the point of delivery of the combination product to the patient (i.e. after release of the medicament contents of the first and at least one further medicament container from isolated containment but prior to patient delivery) and as an integral part of the medicament release/delivery process rather than by a step-wise process involving a defined ‘pre-mixing’ step followed by a separate ‘delivery’ step.

Preferably, the mixing means acts to promote the mixing by making use of the energy (usually, kinetic energy) inherent in the medicament release/delivery process. In one aspect, such energy results from an active medicament release (e.g. firing an MDI or aerosolising a liquid spray product). In another aspect, in the case of an inhaled product, such energy results from the patient inhaling.

In aspects, the action of the mixing means may be tailored to provide an optimum (or minimum) degree of mixing, which may be predetermined or may be set in response to diagnostic input. Turbulent mixing is, in aspects, desirable.

Suitably, the mixing means comprises one or more mechanical mixing promoters, which comprises mechanical features shaped to promote mixing. In aspects, such mixing promoters comprise or consist of baffles, propellers, paddles, vanes and venturi forms.

Suitably, the mixing means comprises a mixing chamber including inlets for receiving medicament from each medicament container and an outlet for delivery of ‘mixed’ combination medicament product to the patient for delivery. Where the combination product is to be inhaled, delivery preferably occurs through a common outlet (e.g. a mouthpiece or nozzle) that communicates with the mixing chamber. The ergonomics of the mixing chamber will be arranged to ensure effective mixing of the separate medicament feeds.

Where a mixing chamber is present its overall form and/or inner surface may be shaped (e.g. with defined surface indentations or protrusions) that promote mixing.

Alternatively or in addition, one or more mechanical mixing promoters may be provided to the mixing chamber.

In one aspect, the mixing chamber is provided with energisation means for energising the mixing process (e.g. by provision of an energised or turbulent airflow).

In another aspect, where the medicament dispenser is an inhalation device, the mixing chamber is arranged (e.g. through shaping or by the provision of particular features thereto) to harness the energy provided by a patient's inward breath to promote the desired mixing. Venturi channelling of the patient air flow by the mixing chamber is envisaged. Helical form channels are envisaged.

In combination, the first medicament and at least one further medicament comprise a defined combination product. That is to say, that when combined together the distinct active medicament doses released by actuation of the device form a dose of a mixed ‘multi-active’ medicament treatment.

On actuation, the dispenser device is designed to deliver a dose portion of the first medicament and a dose portion of each at least one further medicament. The term ‘dose portion’ is employed because in the context of the invention the distinct ‘portions’ are brought together on delivery to form a combination (i.e. multi-active) product dose.

In one particular aspect, the first medicament container contains plural co-formulation compatible medicament components, and each at least one further medicament container contains at least one co-formulation incompatible medicament component.

The term ‘co-formulation compatible’ herein is used to mean compatible in the sense of being amenable to co-formulation, perhaps even displaying synergetic co-formulation characteristics. The term ‘co-formulation incompatible’ is used to mean the reverse, that is to say for whatever reason including chemical or physical incompatibility or simply lack of synergetic characteristics or benefits, the medicament components are either non-amenable to co-formulation or for whatever reason, including for development simplicity, preferably not co-formulated.

In one particular aspect, the medicament dispenser device is designed to receive the first and only one further medicament container. Thus, the device functions as a bi-container medicament dispenser device.

The first and at least one further medicament containers may be of a similar-type or in aspects, be of a different type. This enables additional flexibility in that one container may for example, accommodate a product in dry powder form whereas the other container accommodates product in liquid, solution or aerosol form.

In one aspect, the first medicament container and the at least one further medicament container are of a type adapted to be used with a medicament dispenser selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI). The first medicament dispenser and at least one further remain different in type.

In one aspect, the first medicament dispenser is a reservoir dry powder inhaler (RDPI), and the at least one further medicament dispenser is of a type selected from the group consisting of a multi-dose dry powder inhaler (MDPI), a metered dose inhaler (MDI) and a liquid spray inhaler (LSI).

In another aspect, the first medicament dispenser is a multi-dose dry powder inhaler (MDPI), and the at least one further medicament dispenser is of a type selected from the group consisting of a reservoir dry powder inhaler (RDPI), a metered dose inhaler (MDI) and a liquid spray inhaler (LSI).

In a further aspect, the first medicament dispenser is a metered dose inhaler (MDI), and the at least one further medicament dispenser is of a type selected from the group consisting of a reservoir dry powder inhaler (RDPI), a multi-dose dry powder inhaler (MDPI) and a liquid spray inhaler (LSI).

In a further aspect, the first medicament dispenser is a liquid spray inhaler (LSI), and the at least one further medicament dispenser is of a type selected from the group consisting of a reservoir dry powder inhaler (RDPI), a multi-dose dry powder inhaler (MDPI) and a metered dose inhaler (MDI).

By reservoir dry powder inhaler (RDPI) it is meant an inhaler having a reservoir form container pack suitable for containing multiple (un-metered doses) of medicament product in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose container pack containing (or otherwise carrying) multiple, define doses (or parts thereof of medicament product. In a preferred aspect, the carrier has a blister pack form, but it could also, for example, comprise a capsule-based pack form or a carrier onto which medicament has been applied by any suitable process including printing, painting and vacuum occlusion.

In one aspect, the multi-dose pack is a blister pack comprising multiple blisters for containment of medicament product in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 in the name of Glaxo Group Ltd describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

By liquid spray inhaler (LSI) it is meant a medicament dispenser suitable for dispensing medicament in spray form, wherein the medicament is typically formulated in liquid or solution form and comprised in a liquid container. The container is typically provided with a means of metering to a spray generator, which imparts energy to the liquid or solution, thereby generating a spray for inhalation by the patient. The spray generator, in aspects, comprises a vibrating element (e.g. a mesh) that provides vibrational energy to the formulation, thereby resulting in its aerosolisation. In other aspects, the spray generator comprises a pump mechanism, which either delivers the medicament directly to the patient (as a liquid spray) or which delivers the medicament to an intermediate position at which further energy is supplied thereto to further propel, aerosolise or otherwise direct the medicament dose to the patient.

The medicament dispenser device herein has unitary form, and typically has a housing shaped to receive, and enable the release of medicament product from the first and at least one further medicament containers.

In one aspect, the housing integrally comprises a release means for releasing medicament from at least one, preferably all of the medicament dispensers. Suitably, the release means for each medicament container is coupled, thereby enabling simultaneous delivery of medicament from each dispenser in response to a single patient actuation step.

In another aspect, the housing is shaped to receive the medicament containers, each of which is provided with respective release means. In this case, the release means have typically been adapted for receipt by the housing. The medicament dispenser and release means therefor are in one aspect, supplied as independently operable ‘cassette refills’ for the unitary device.

The medicament dispenser device may be provided with means for varying the amount of medicament product released from each medicament container. Customized delivery of combination medicament product may therefore be achieved through varying the relative ratios of each individual medicament product delivered as well as by varying the absolute amount of medicament product delivered. Variable timing mechanisms are envisaged for achieving such customisation.

Delivery of the combination product (e.g. after mixing) to the patient is preferably through a single outlet. The outlet is typically positioned to be in communication with the distinct medicament dose portions delivered. The outlet may have any suitable form. In one aspect, it has the form of a mouthpiece and in another it has the form of a nozzle for insertion into the nasal cavity of a patient.

The outlet is preferably a single outlet, which communicates with the distinct medicament dose portions delivered via a common air channelling means (e.g. formed as an air-pipe or common manifold). The patient may therefore breathe in through a single outlet, and that breath be transferred through the common channelling means to (all of the released medicament dose portions, thereby enabling their inhalation as a multi-active combined product.

Any or all mechanical components of the device may be driven by either an electronic or mechanical drive system or combination thereof.

Suitably electronic drive means typically comprise a motor, preferably an electrically-powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated.

Ultrasonic motors are well known in the art and are commercially available (e.g. BMSTU Technological Cooperation Centre Ltd, Moscow, Russia; Shinsei Corporation, Tokyo, Japan).

Ultrasonic motors do not use coils or magnets but comprise a piezo-electric ceramic stator which drives a coupled rotor. The stator generates ultrasonic vibrations which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the device further comprises actuating means for actuating said electronic drive system. Said actuating means may take the form of a switch, push-button, or lever.

Suitably, the medicament dispenser device is provided with at least one actuation indicator associated with the first medicament container and the at least one further medicament container. The association may be direct, or it may be through some form of intermediary component such as a coupling component.

The term ‘actuation indicator’ is used herein to mean any means for indicating, or in particular counting, when the dispenser device is actuated. That indication may be based on detection of any actuation step, which will result in delivery of medicament from the dispenser device or it may be based on detection of the medicament released by an actuation step.

The actuation indicator particularly includes means for registering and displaying dose release or dose count information to the patient. At a basic level, that information may simply relate to the fact that an actuation step or medicament release has been detected, but more often the information relates to the number of doses delivered or remaining of each medicament in the dispenser device. The information may be delayed in digital or analogue form, typically using standard count indicia (e.g. ‘999’ to ‘000’ indicia count display). Embodiments involving either ‘counting up’ or ‘counting down’ in increments are envisaged.

Dose release or dose count information may be displayed for the ‘combined product’ (i.e. first and at least one further medicament) together, or it may be separately displayed for each separate medicament component of the combination.

In aspects, the actuation indicator comprises an actuation sensor. The actuation sensor is for example, sensitive to parameters selected from the group consisting of electro magnetic radiation, magnetic field, light, motion, temperature, pressure, sound, oxygen concentration, carbon dioxide concentration and moisture. The actuation sensor is arranged to sense the actuation of the dispenser. In one aspect, the actuation sensor is integral with a housing of the medicament dispenser device, for example moulded into a housing of the dispenser device or attached thereto. Alternatively, the actuation sensor is reversible attachable to the housing.

Where release of medicament is to be detected, the actuation indicator suitably comprises a release sensor for directly detecting the medicament release. The positioning of the release sensor in the dispenser device will be arranged to maximise detection of each, whilst minimising any interference effects (including those due to release of other medicament) and whilst minimising any effect on the delivery of each medicament to the patient.

The actuation indicator may be associated mechanically or electronically with the actuation or release sensor(s), such that when the detector detects actuation or medicament release a signal is sent to the actuation indicator to record that a (part) dose has been dispensed.

Suitably, the actuation indicator additionally comprises a visual display unit for display of the data. Preferably, the visual display unit displays the number of doses of medicament used or remaining within the container. Preferably the doses are displayed numerically, by a series of coloured lights or by a monochrome bargraph.

In one aspect, the medicament dispenser device includes an electronic control system for controlling the release of contents from the first and at least one further medicament container. The electronic control system may have any suitable form and incorporate any of the electronic system aspects as described hereinafter.

In one aspect, the electronic control system is responsive to inputs directly provided to it by an individual such as for example, a medical professional (e.g. G.P.), a pharmacist or the patient. In this aspect, any tailoring of the composition of the combination product is determined by these inputs. In one particular aspect, the inputs are set (or even, pre-set) at particular time such as at the prescription of the dispenser to the patient.

In another aspect, the electronic control system is associated with or responsive to a patient diagnostic system that collects diagnostic information relating to the patient's current disease condition. Tailoring of the composition or desired degree of mixing of the combination product is therefore determinable by reference to diagnostic data gathered and processed by this system.

Where the dispenser is an inhaler for dispensing medicament for the relief of respiratory disorders, examples of suitable diagnostic data would include diagnostics related to the patient's physical breath characteristics including particularly breath cycle data or peak flow or FEV-1 data.

Suitably, the device additionally comprises an electronic data management system. The electronic data management system has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body. Alternatively, the electronic data management system forms part of a base unit which is reversibly associable with the body.

Suitably, the device additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the device to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the device additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

In one aspect, the device includes an electronic dose reminder system. This may be configured to have any suitable form and may be powered by mains, stored (e.g. battery) or self-regenerating (e.g. solar) energy power source.

The electronic dose reminder system comprises an electronic timer for timing an elapsed time period corresponding to the time since the last actuation of the device; a dose interval memory for storing data relating to a prescribed dose interval time period; and a patient alerter for alerting a user. The alerter activates when the elapsed time period exceeds the prescribed dose interval time period.

The electronic timer progressively times the period since the last actuation of the device (the ‘elapsed time period’). The timer can have any suitable electronic form. The significance of the ‘elapsed time period’ is that in use, it typically corresponds to the time elapsed since the previous dose delivery event.

The timer may be configured to include an automatic re-zeroing feature such that on subsequent actuation of the device the timer count starts again from zero.

The dose interval memory stores data relating to a prescribed dose interval time period. By way of examples, if the medicament is to be taken twice a day at a regular interval, the prescribed dose interval may be set as twelve hours, or for a once daily treatment the value may be set at twenty four hours. In aspects, the system may be configured to allow for ready readjustment of the prescribed dose interval time period, or it may be configured in secure fashion such that any readjustment may be made only by a designated prescriber (e.g. a medical professional or pharmacist). Password and/or other security means may be employed. The prescribed dose interval may be configured to be variable over a particular course of treatment, or alternatively it may be fixed at a set dose interval over the full course of treatment.

The patient alerter is designed to communicate an alert to the user. The alerter activates only when the holding time period exceeds the prescribed dose interval time period. By way of an example, for a once daily treatment with a prescribed dose interval of twenty four hours, the alerter would activate only when the holding time period, as timed by the electronic timer, exceeds twenty four hours since at this point another dose is due to be taken. It may thus, be appreciated that the alerter acts functionally as a reminder to the patient that a dose is due to be taken.

The alerter may in aspects, comprise a visual device, such as a liquid crystal display (LCD) or an array of light-emitting diodes (LEDs), connected to a battery-driven timing device of any convenient kind known to those skilled in the art. The visual device may be configured to display information such as the actual time or the elapsed time from the taking of a previous dosage and may have superimposed thereon additional messages, such as a textual instruction to take a dose of the medicament. Alternatively, the instruction to take the medicament may be conveyed merely by displaying a warning colour or by causing the display to flash or in any other way.

In a further alternative arrangement, no specific time or elapsed time information is displayed, but the alerter merely provides a warning signal that indicates the necessary action to the user.

Depending upon the lifestyle of the user, additional or alternative warnings may be of greater assistance than purely visual warnings. Accordingly, the invention envisages that the alerter may provide audible and/or tactile warnings, such as vibration, instead of (or in addition to) visual warnings.

The alerter may provide a single, one-off alert. More preferably, the alerter is configured to provide the alert over a set period of time (the ‘alerting time period’ or ‘alerting window’). In one aspect, the alerting time period is calculated as a function of (e.g. fraction of) the dose interval time period. For example, for a twice daily treatment with a dose interval time period of twelve hours, the alerting time period may be set as half that period (i.e. six hours). In this case, the alert is then provided for the six hours immediately following the activation of the alert.

The system is typically configured such that the alerting signal cuts off when the user removes the medicament delivery device from the holder to enable dosing of medicament therefrom. The system is then reset. Other manual cutoffs/overrides may also be included.

In a subtle aspect of the present invention, it may be appreciated that the relevant timeframe for detecting, timing and alerting are determined by user action in relation to the system, and in particular by user action. The dose reminder capability is therefore independent of any particular defined external time zone (e.g. the local time zone relative to Greenwich Mean Time, as defined by the twenty four hour clock) because the user action defines its own ‘reminder timeframe’. This provides advantages over other known reminder systems, which are reliant on user reference to defined external time frames. The advantage is particularly great for the international traveller since complex calculations involving different local time zones are avoided.

It will be appreciated from the above description that the various components of the electronic dose reminder system interrelate with each other to provide the required functionality. The system may be configured in any suitable fashion using known electronic components and circuitry methods.

Suitably, the device additionally comprises an actuation detector for detecting actuation of any one of the medicament dispensers thereof wherein said actuation detector transmits actuation data to the electronic data management system.

The device may additionally comprise a safety mechanism to prevent unintended multiple actuations of the component medicament dispensers. The patient is thereby, for example, protected from inadvertently receiving multiple doses of medicament in a situation where they take a number of short rapid breaths. More preferably, the safety mechanism imposes a time delay between successive actuations of the release means. The time delay is typically of the order of from three to thirty seconds.

Suitably, the device additionally comprises a release detector for detecting release of medicament from the cassette, wherein said release detector transmits release data to the electronic data management system.

Suitably, the device additionally comprises a shake detector for detecting shaking of the medicament container (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser device is actuable in response to the inward breath of a patient and includes a breath sensor of an y suitable type (e.g. mechanical or electronic) for detecting that inward breath wherein the sensor communicates with the electronic control system. Thus, in use the patient breathes in through the dispenser (e.g. through the mouthpiece); the breath is detected by the breath sensor; the sensor communicates with the electronic control system to convey an ‘inward breath detected’ signal; and the electronic control system responds by releasing medicament from one or more of the medicament containers for inhalation by the patient.

In one aspect, the breath sensor comprises a breath-movable element that is movable in response to the breath of a patient. Preferably, the breath-movable element is selected from the group consisting of a vane, a sail, a piston and an impeller.

In another aspect, the breath sensor comprises a pressure sensor for sensing the pressure profile associated with the breath of a patient. In a further aspect, the breath sensor comprises an airflow sensor for sensing the airflow profile associated with the breath of a patient. In a further aspect, the breath sensor comprises a temperature sensor for sensing the temperature profile associated with the breath of a patient. In a further aspect, the breath sensor comprises a moisture sensor for sensing the moisture profile associated with the breath of a patient. In a further aspect, the breath sensor comprises a gas sensor for sensing the oxygen or carbon dioxide profile associated with the breath of a patient. In a further aspect, the breath sensor comprises a piezoelectric or piezoresistive element.

Suitably, the device additionally comprises a breath trigger for triggering release from one or all of the component medicament containers, said breath trigger for example, being actuable in response to a trigger signal from the electronic data management system. Preferably, the electronic data management system includes a predictive algorithm or look-up table for deriving from the breath data when to transmit the trigger signal. For example, a real-time analysis of the patient breath waveform may be made and the trigger point derived by reference to that analysed waveform.

In one aspect, the medicament dispenser herein includes a timing control system for controlling the time of release of contents from the first and at least one further medicament container. The timing control system, generally communicates with the electronic control system with which it may in aspects, form an integral part.

The timing control system is suitably arranged to vary the relative time of release of each medicament component from its respective medicament container. Each medicament component may therefore be arranged for simultaneous or sequential release, although in general where components are released sequentially the time delay between releases of each separate medicament component is short (e.g. milliseconds) to ensure that a combined product is provided for administration to the patient.

In a further aspect, by varying the time of release, the ratio of quantity of each medicament component released can also be varied, thereby enabling the provision and delivery of ‘tailored’ combined products.

The timing control system generally comprises electronic components and is arranged to be responsive to the electronic control system. In aspects, the timing control system is arranged to be responsive to a diagnostic system, which is arranged to diagnose patient disease characteristics and thereby select and deliver and suitable tailored combined product dose.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the device additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the device comprises in association with a body or housing thereof, a first transceiver for transmitting and receiving data and in association with the medicament container, a second transceiver for transmitting and receiving data, wherein data is transferable in two-way fashion from the first transceiver to the second transceiver. The data is preferably in digital form and suitable for transfer by electronic or optical means. A medicament dispenser of this general type is described in pending UK Patent Application No. 0020538.5.

One advantage of embodiments of this type is the ability to store many types of information in different parts of the memory structure of the transceivers. The information is furthermore stored in a form which is readily and accurately transferable. The information could for example, include manufacturing and distribution compliance information written to the memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory which uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the device with a medicament dispenser or ‘refill’ the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within any medicament container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the device. Or alternatively, data may be stored in a database memory of the electronic data management system and periodically downloaded to any transceiver. In either case, a history of the usage of the device may be built up in the memory of a transceiver.

In one embodiment herein, a history of the usage of the device is transferred to the second transceiver. When the medicament carriers in the cassette are exhausted it is exchanged by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patient is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure which allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly-powered and the term passive is used to mean indirectly-powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver. Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is mouldable or weldable to the cassette or housing. Suitably, the carrier encases the tag. More preferably, the carrier forms a hermetic seal for the tag.

In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radiofrequency, magnetic, optical and laser form. Any suitable channels may be used to channel the energy including fibre optic channels.

In one aspect, the second transceiver comprises a radiofrequency identifier comprising an antenna for transmitting or receiving radiofrequency energy; and an integrated circuit chip connecting with said antenna, and the first transceiver comprises a reader for said radiofrequency identifier. In this case the radiofrequency identifier is a passive transceiver and the reader is an active transceiver. An advantage of radiofrequency identifier technology is that the reader need not be in direct contact with the radiofrequency identifier tag or label to be read.

The radiofrequency identifier can be any known radiofrequency identifier. Such identifiers are sometimes known as radiofrequency transponders or radiofrequency identification (RFID) tags or labels. Suitable radiofrequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radiofrequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magnetorestrictive element which is readable by application of an interrogating alternating magnetic field in the presence of a magnetic bias field which results in resonance of the magnetorestrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet which may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit which is reversibly associable with the body. The base unit may for example, form a module receivable by the body such as a snap-in module.

Suitably, the device additionally comprises a communicator for wireless communication with a network computer system to enable transfer of data between the network computer system and the electronic data management system.

Suitably, the data is communicable between the network computer system and the electronic data management system in encrypted form. All suitable methods of encryption or partial encryption are envisaged. Password protection may also be employed. Suitably, the communicator employs radiofrequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radiofrequency signals. A suitable spread spectrum protocol is the Bluetooth (trade mark) standard which employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entrypoint including an entrypoint managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet, which may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctors practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the device additionally comprises a geographic positioning system such as a global positioning system or a system, which relies on the use of multiple communications signals and a triangulation algorithm.

The constituent medicaments of the plural medicament dose portions suitably, in combination comprise a combination medicament product. Suitably the medicaments are selected from the group consisting of albuterol, salmeterol, fluticasone propionate and beclomethasone dipropionate and salts or solvates thereof. Preferably, the combination comprises salmeterol xinafoate and fluticasone propionate.

### BRIEF DESCRIPTION OF THE DRAWINGS

The invention will now be described with reference to the accompanying drawings in which:

FIG. 1a shows a sectional plan view of a medicament dispenser device in accord with the invention;

FIG. 1b shows a perspective view of a detail of the medicament dispenser device of FIG. 1a;

FIGS. 1c to 1h show side views of different forms of mixing promoter suitable for use with the medicament dispenser device of FIG. 1a;

FIG. 2a shows a sectional plan view of a second medicament dispenser device in accord with the invention;

FIGS. 2b and 2c show sectional side views of variations to modification of a manifold suitable for use with the second medicament dispenser device of FIG. 2a;

FIG. 3 shows a sectional side view of a manifold and mouthpiece suitable for use in a variation of the medicament dispenser device of FIG. 1a;

FIG. 4a shows a perspective view of a dual MDI dispenser herein;

FIG. 4b shows the dispenser of FIG. 4a in part cut-away view; and

FIGS. 4c to 4e show cross-sectional views of different modifications of the common outlet passage 412 of the dual MDI dispenser device of FIGS. 4a and 4b in accord with the present invention.

### DETAILED DESCRIPTION OF THE DRAWINGS

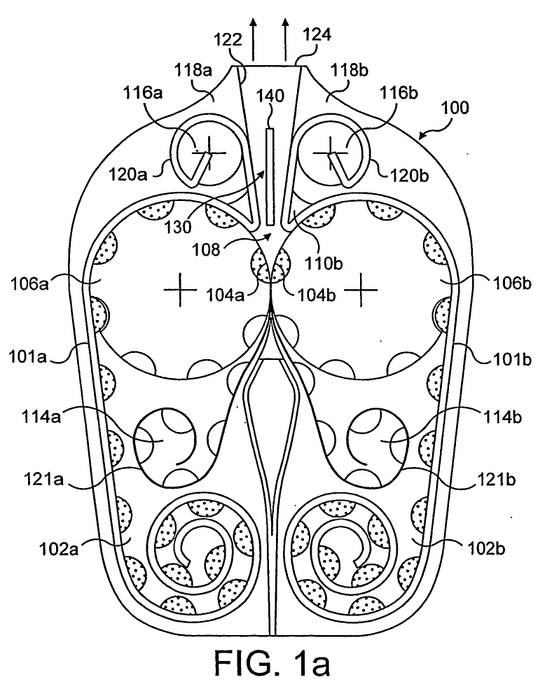

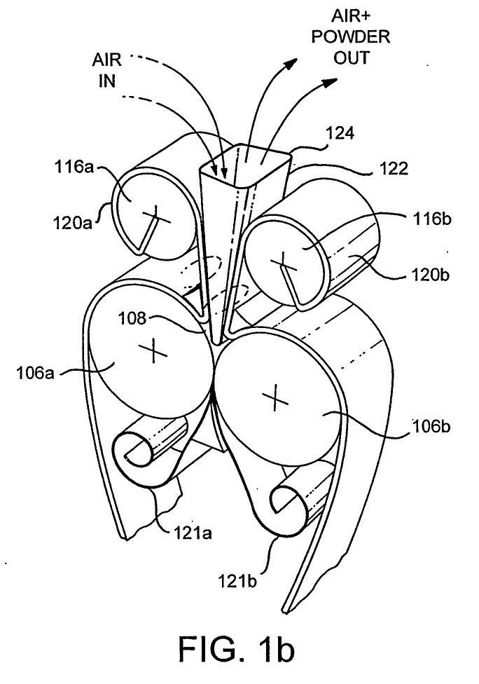

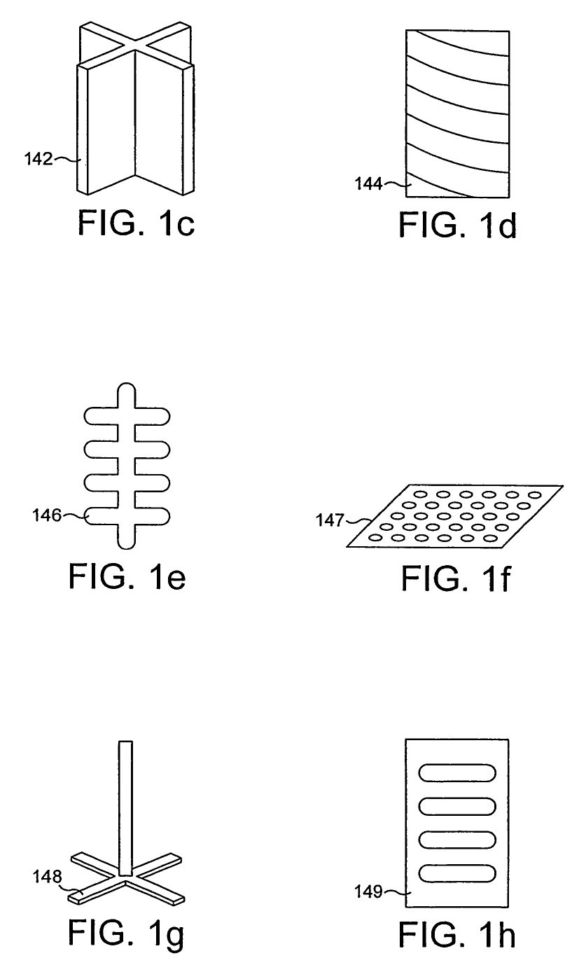

FIG. 1a illustrates the base unit 100 of a medicament dispenser according to the invention. In use, a cover (not shown) would be provided to the base unit 100. First and second medicament-containing blister strips 101a, 101b are positioned within respective left and right chambers 102a, 102b of the base unit 100. Each blister strip 101a, 101b engages a respective multi-pocket index wheel 106a, 106b, and successive pockets are thereby guided towards a commonly located opening station 108. The rotation of the index wheels 106a, 106b is coupled. At the opening station 108, the lid foil 120a, 120b and base foil 121a, 121b parts of each strip 101a, 101b are peelably separable about a beak 110a, 110b. The resulting empty base foil 121a, 121b coils up in respective base take-up chambers 114a, 114b. The used lid foil 120a, 120b is fed over its respective beak 110a, 110b and coiled about a lid take-up spindle 116a, 116b in the lid take-up chamber 118a, 118b.

Released powder form medicament from opened pockets 104a, 104b of both the first 101a and second 101b strips is accessible via manifold 122 to the common outlet 124 for inhalation by the patient. The manifold 122 defines a mixing chamber area 130 into which the released powders travel for mixing thereof prior to delivery at the common outlet 124. The mixing chamber 130 is provided with a baffle 140 to promote turbulent flow and enhance mixing of the released powders. Importantly, the dispenser thereby enables different medicament types to be stored separately in each of the strips 101a, 101b but the release and delivery thereof to the patient as a ‘mixed’ multi-active combined inhaled product.

FIG. 1b shows the release of medicament in more detail. The patient breathes in through the outlet 124 resulting in negative pressure being transmitted through manifold 122 to the opened pockets of the strips 101a, 101b at the opening station 108. This results in the creation of a venturi effect which results in the powder contained within each of the opened pockets 101a, 101b being drawn out through the common manifold 122 to the mixing chamber 130 and baffle 140 therein for mixing and thence to the common outlet 124 for inhalation by the patient. Mixing of each separately delivered component of the combined medicament product thus happens as an integral part of the release/delivery process.

In aspects, the baffle 140 of FIG. 1a may be replaced by other forms of mechanical mixing promoter as shown in FIGS. 1c to 1h. Thus, FIG. 1c shows a mixing promoter of elongate cruciform 142 structure. FIG. 1d shows a mixing promoter of screw thread 144 form. FIG. 1e shows a mixing promoter 146 having a multi-branched structure. FIG. 1f shows a mesh-form mixing promoter 147. FIG. 1g shows a propeller-shaped mixing promoter 148. FIG. 1h shows a slatted paddle form of mixing promoter 149. Embodiments are envisaged in which the mixing promoter or any combinations of two or more mixing promoters is arranged in any suitable geometric arrangement. Optimisation of the form and positioning of the mixing promoter(s) would be by workshop modification.

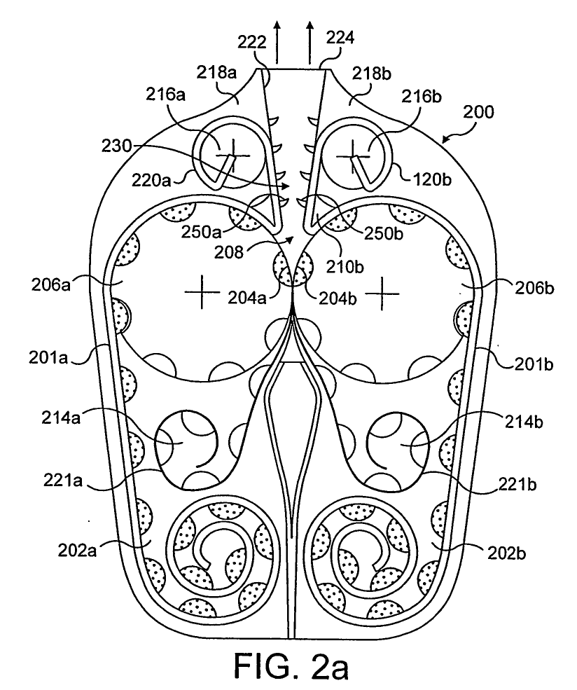

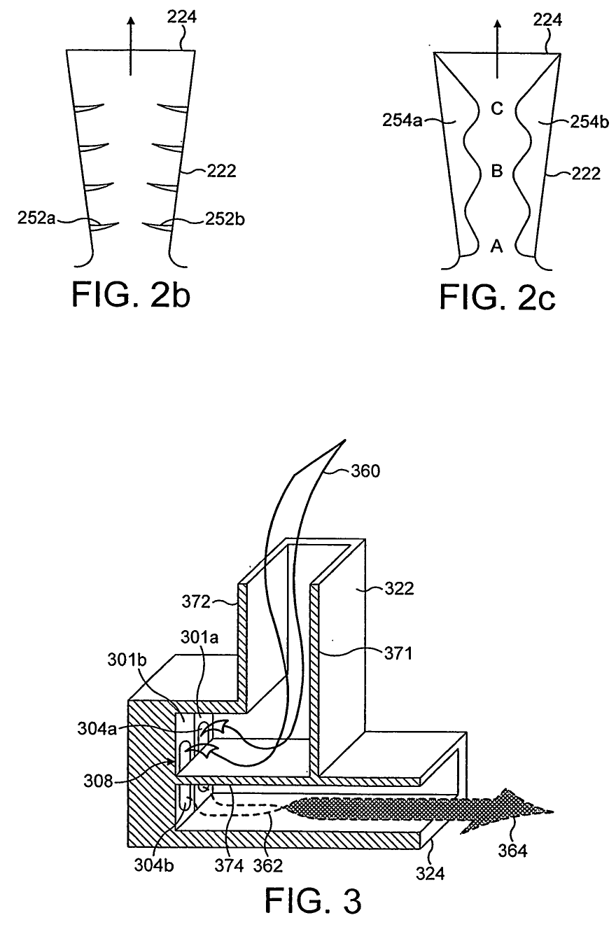

FIG. 2a illustrates the base unit 200 of a second medicament dispenser device according to the invention. It will be appreciated that this device closely relates to the device of FIG. 1a except that the mixing means is varied. In essence, the baffle 140 of FIG. 1a is replaced by surface modification of the manifold 222, which defines the mixing chamber 230 in FIG. 2a.

In more detail, first and second medicament-containing blister strips 201a, 201b are positioned within respective left and right chambers 202a, 202b of the base unit 200, to which a cover is provided, in use. Each blister strip 201a, 201b engages a respective multi-pocket index wheel 206a, 206b, and successive pockets are thereby guided towards a commonly located opening station 208. Rotation of the index wheels 206a, 206b is coupled. At the opening station 208, the lid foil 220a, 220b and base foil 221a, 221b parts of each strip 201a, 201b are peelably separable about a beak 210a, 210b. The resulting empty base foil 221a, 221b coils up in respective base take-up chambers 214a, 214b. The used lid foil 220a, 220b is fed over its respective beak 210a, 210b and coiled about a lid take-up spindle 216a, 216b in the lid take-up chamber 218a, 218b.

Released powder form medicament from opened pockets 204a, 204b of both the first 201a and second 201b strips is accessible via manifold 222 to the common outlet 224 for inhalation by the patient. The manifold 222 defines a mixing chamber area 230 into which the released powders travel for mixing thereof prior to delivery at the common outlet 224. The surface of the manifold 222 is provided with plural jagged teeth 250a, 250b, which acts such as to promote turbulent flow within the mixing chamber 230 and thereby enhances mixing of the released powders.

In use, the patient breathes in through the common outlet 224 resulting in negative pressure being transmitted through manifold 222 to the opened pockets of the strips 201a, 201b at the opening station 208. This results in the creation of a venturi effect which results in the powder contained within each of the opened pockets 201a, 201b being drawn out through the common manifold 222 and experiencing the jagged teeth thereof 250a, 250b in the mixing chamber 230 for mixing and thence to the common outlet 224 for inhalation by the patient. Mixing of each separately delivered component of the combined medicament product thus happens as an integral part of the release/delivery process.

It will be appreciated that in variations of the medicament dispenser device of FIG. 2a other surface modifications of the manifold 222 may be employed to create different flow patterns and hence to enhance mixing.

In one variation, shown in FIG. 2b plural resilient, hair-like protrusions 252a, 252b are provided to the manifold 222. In another variation shown in FIG. 2c, undulating form inserts 254a, 254b are provided, wherein the effect of the inserts is to create plural points A, B, C of constricted flow along the air path from opened medicament-containing pockets (not visible) to the common outlet 224.

FIG. 3 illustrates a manifold and mouthpiece design suitable for use in a variation of a medicament dispenser device of the type shown in FIG. 1a.

First and second medicament components of the combination medicament product for delivery are contained within blister pockets 304a, 304b of two elongate blister strips 301a, 301b. In common with the device of FIG. 1a, at common opening station 308, the opened pockets 304a, 304b are exposed to an air path 360 (created in response to the inward breath of a patient), which flows from air inlet 323. In the variation of FIG. 3, the air path is channelled through the opened pockets 304a, 304b to aerosolise the powdered medicament products contained respectively therein and thence to transport the aerosolised powder product 364 to common outlet 324 for patient inhalation thereof. Mixing is at mixing area 362 and thus occurs prior to flow of the combination product 364 from the common outlet.

It will be appreciated that the form of manifold 322 is crucial to the correct channelling of the air path of the device of FIG. 3a. In more detail, the incoming air path 360 is channelled first by vertical (as shown) walls 371, 372 to bisecting wall 374, which is located such as to bisect the two opened pockets 304a, 304b thereby enabling air flow into one side of each opened pocket 304a, 304b and flow of air and aerosolised powder from out of the other side of each pocket 304a, 304b. The aerosolised powder is then channelled to the mixing area 362 and finally to the common outlet 324 for patient inhalation, as previously described.

In variations of the design of FIG. 3, various baffles and other forms of mixing promoter may be incorporated as described in FIGS. 1a to 1h. Alternatively, the surface of the manifold 322 may be modified to include suitable shaping, as described in FIGS. 2a to 2c.

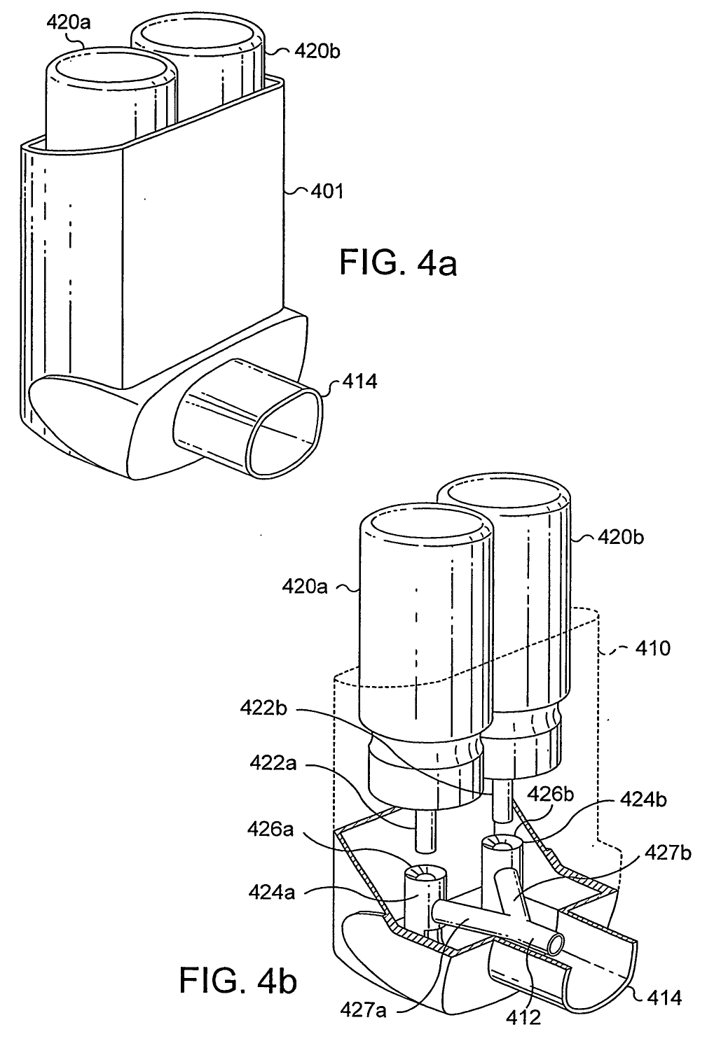

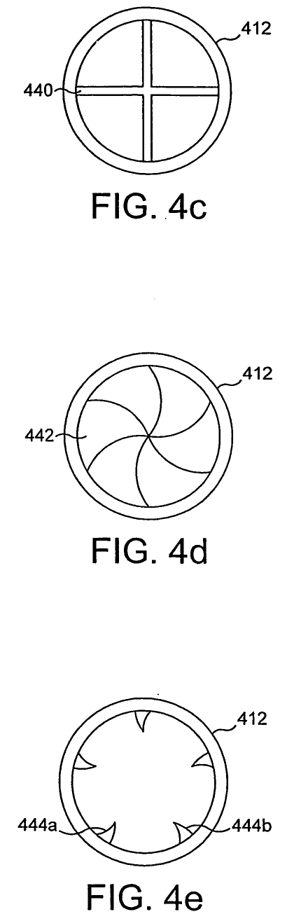

FIGS. 4a and 4b show a dual MDI dispenser device, which is modified in accord with the invention as shown in FIGS. 4c to 4e.

FIG. 4a shows a perspective view of a dual MDI dispenser herein and FIG. 4b shows the dispenser of FIG. 4a in part cut-away view. The dual MDI dispenser comprises a generally L-shaped tubular housing 401 shaped for receipt of two aerosol containers 420a, 420b. The first aerosol container 420a contains first active medicament component in aerosol formulation form. The second aerosol container 420b contains second active medicament component in aerosol formulation form. The housing is open at one end (which will hereinafter be considered to be the top of the device for convenience of description) and is closed at the other. A common mouthpiece 414 leads laterally from the closed end of the housing 401. In variations, the mouthpiece 414 may if desired, be designed as a nozzle for insertion into the patient's nostril.

Each aerosol container 420a, 420b has an outlet valve stem 422a, 422b at one end. Each valve 422a, 422b can be depressed to release a metered dose from its respective aerosol container 420a, 420b. Each aerosol container 420a, 420b locates in the housing 401 such that one end protrudes from its open top. Valve support 424a, 424b are provided at the lower end of the housing 401 and are provided with respective passages 426a, 426b in which the valve stem 422a, 422b of each respective aerosol container 420a, 420b can be located and supported. A second passage 427a, 427b leads from each support 424a, 424b and is directed towards common outlet passage 412.

In use, the protruding portion of each aerosol container 420a, 420b is depressed to move that container 420a, 420b relative to its valve stem 422a, 422a to open the valve and discharge an aerosol form dose of medicament through passages 427a, 427b to common outlet passage 412 and thence to the mouthpiece 414 from which it can be inhaled by a patient. A measured amount (e.g. a part-combination dose) will be released from each aerosol container 420a, 420b each time it is fully depressed.

It will be appreciated that the required depression of each aerosol container 420a, 420b is achievable by a dual fingered patient action whilst the base of the housing 401 is held in a patient's cupped hand. In variations, the movement of the containers 420a, 420b may be coupled (e.g. through use of a coupling element) thereby ensuring that both containers 420a, 420b are fired in tandem.

The common outlet passage 412 of the dual MDI dispenser device of FIGS. 4a and 4b is modified to incorporate mixing means in accord with the invention as shown in FIGS. 4c to 4e.

In the embodiment of FIG. 4c, the common outlet passage 412 is provided with a cruciform insert 440 to agitate the flow of each aerosol form medicament product and hence, to promote the formation of a mixed combination product.

In the embodiment of FIG. 4d, the common outlet passage 412 is provided with a propeller-shaped insert 440 to provide an angular momentum component to the flow of each aerosol form medicament product and hence, to promote the formation of a mixed combination product.

In the embodiment of FIG. 4c, the common outlet passage 412 is provided with a plural jagged teeth 444a, 444b to agitate the flow of each aerosol form medicament product and hence, promote the formation of a mixed combination product.

In a first particular set of examples, the first medicament active component of each medicament dispenser device shown the previously described Figures is salmeterol xinafoate and the second medicament active component is fluticasone propionate.

In a second particular set of examples, the first medicament active component of each medicament dispenser device shown the previously described Figures is formoterol fumarate and the second medicament active component is fluticasone propionate.

In a third particular set of examples, the first medicament active component each medicament dispenser device shown the previously described Figures is formoterol fumarate and the second medicament active component is beclomethasone dipropionate.

It may be appreciated that any of the parts of the device or any medicament thereof which contacts medicament may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The device of the invention is suitable for dispensing medicament combinations, particularly for the treatment of respiratory disorders such as asthma and chronic obstructive pulmonary disease (COPD), bronchitis and chest infections.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred components of the combinations comprise medicaments selected from albuterol, salmeterol, fluticasone propionate and beclomethasone dipropionate and salts or solvates thereof, e.g., the sulphate of albuterol and the xinafoate of salmeterol.

Preferred components of combinations of active ingredients contain a bronchodilator in combination with an anti-inflammatory. The bronchodilator is suitably a beta-agonist, particularly a long-acting beta-agonist (LABA). Suitable bronchodilators include salbutamol (e.g., as the free base or the sulphate salt), salmeterol (e.g., as the xinafoate salt) and formoterol (eg as the fumarate salt). The anti-inflammatory is suitably an anti-inflammatory steroid. Suitably anti-inflammatory compounds include a beclomethasone ester (e.g., the dipropionate), a fluticasone ester (e.g., the propionate) or budesonide or any salt or solvate thereof. One preferred combination of components comprises fluticasone propionate and salmeterol, or any salt or solvate thereof (particularly the xinafoate salt). A further combination of components of particular interest is budesonide and formoterol or any salt or solvate thereof (e.g. formoterol as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

Aerosol formulations suitable for use with metered dose inhaler (MDI) dispensers typically comprise a propellant. Suitable propellants include P11, P114 and P12, and the CFC-free hydrofluoroalkane propellants HFA-134a and HFA-227.

The MDI aerosol formulation may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the MDI aerosol formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise the part or all of the medicament component, such formulations being commonly referred to as solution formulations.

A surfactant may also be employed in the MDI aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

The device of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief e.g. inhaled morphine.

Accordingly, there is provided the use of a device according to the invention for the treatment of a respiratory disorder, such as asthma and COPD. Alternatively, the present invention provides a method of treating a respiratory disorder such as, for example, asthma and COPD, which comprises administration by inhalation of an effective amount of medicament product as herein described from a device of the present invention.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A medicament dispenser device for use in the delivery of a combination medicament product, the device comprising:
a first medicament container for containing a first medicament component;
first release means for releasing the contents of said first medicament container;
at least one further medicament container for containing at least one further medicament component;
at least one further release means for releasing the contents of each said at least one further medicament container; and
mixing means for promoting the mixing of the released contents of the first and at least one further medicament container,
wherein the first medicament component is kept separate from the at least one further medicament component until the point of release thereof for delivery in combination.

2. A medicament dispenser device according to claim 1, wherein said mixing means promotes mixing of said released contents immediately prior to delivery in combination thereof.

3. A medicament dispenser device according to claim 1, wherein the mixing means promotes mixing of the released contents as an integral part of the release and delivery in combination thereof.

4. A medicament dispenser device according to claim 1, wherein the mixing means promotes of the released contents by making use of the energy inherent in the release and delivery in combination thereof.

5. A medicament dispenser device according to claim 1, wherein the mixing means promotes mixing of the released contents by making use of energy input by the patient.

6. A medicament dispenser device according to claim 1, wherein the mixing means provides for turbulent mixing of the released contents.

7. A medicament dispenser device according to claim 1, wherein the mixing means comprises one or more mechanical mixing promoters comprising mechanical features arranged to promote mixing.

8. A medicament dispenser device according to claim 7, wherein said one or more mechanical mixing promoters comprise mechanical features selected from the group consisting of baffles, propellers, paddles, vanes and venturi.

9. A medicament dispenser device according to claim 1, wherein the mixing means comprises a mixing chamber including an inlet for receiving released medicament contents from each medicament container and a common outlet for delivery of combination medicament product to the patient for delivery.

10. A medicament dispenser according to claim 9, wherein the inner surface of the mixing chamber is shaped to promote mixing.

11. A medicament dispenser according to claim 10, wherein the inner surface of the mixing chamber is provided with one or more indentations or protrusions.

12. A medicament dispenser device according to claim 1, wherein the mixing means is provided with energisation means for energising the mixing of the released contents.

13. A medicament dispenser device according to claim 9, in the form of an inhalation device wherein, the mixing chamber is arranged to harness the energy provided by a patient's inward breath to promote mixing of the released contents.

14. A medicament dispenser device according to claim 13, wherein the mixing chamber provides for venturi channelling of said patient's inward breath.

15. A medicament dispenser device according to claim 1, wherein the device comprises the first medicament container and only one further medicament container.

16. A medicament dispenser device according to claim 1, wherein the device includes at least one actuation indicator associated with the first medicament container the at least one further medicament container.

17. A medicament dispenser device according to claim 1, wherein the device includes a timing control for controlling the relative time of release of contents from the first and at least one further medicament container.

18. A medicament dispenser device according to claim 17, wherein the first medicament container contains said first medicament and the at least one further medicament container contains said at least one further medicament.

19. A medicament dispenser device according to claim 18, wherein the first medicament comprises a bronchodilator and the at least one further medicament comprises an anti-inflammatory.

20. A medicament dispenser device according to claim 19, wherein said bronchodilator is a beta-agonist and said anti-inflammatory is a steroid.

21. A medicament dispenser device according to claim 20, wherein said bronchodilator is selected from the group consisting of salbutamol, salmeterol, formoterol and any salts or solvates thereof and mixtures thereof.

22. Medicament dispenser device according to claim 20, wherein said anti-inflammatory is selected from the group consisting of a beclomethasone ester, fluticasone ester, budesonide and any salt or solvates thereof and mixtures thereof.

23. (canceled)

24. A method of treating a respiratory disorder in a patient in need thereof comprising administering an effective amount of medicament suitable for treating said respiratory disorder to the patient by inhalation from a medicament dispenser according to claim 1.
